(19) United States
(12) Patent Application Publication
(10) Pub. No.: US 2003/0229390 A1
(43) Pub. Date: Dec. 11, 2003

(54) On-stent delivery of pyrimidines and purine analogs

(75) Inventors: Paul Ashton, Boston, MA (US); Jianbing Chen, Belmont, MA (US)

Correspondence Address: ROPES & GRAY LLP, ONE INTERNATIONAL PLACE, BOSTON, MA, 02110-2624, US

(73) Assignee: Control Delivery Systems, Inc., Watertown, MA

(21) Appl. No.: 10/401,039

(22) Filed: Mar. 27, 2003

Related U.S. Application Data
(63) Continuation-in-part of application No. 10/245,840, filed on Sep. 17, 2002.
(60) Provisional application No. 60/322,428, filed on Sep. 17, 2001.
(60) Provisional application No. 60/372,761, filed on Apr. 15, 2002.

(51) Int. Cl. A61F 2/06

(52) U.S. Cl. 623/1.15; 623/1.42; 424/423

(57) ABSTRACT

Disclosed is a sustained release system that includes a polymer, a nucleoside analog or prodrug or codrug thereof, and optionally one or more additives that reduce the rate of release of the drug from the polymer. The device using such inventive system permits improved drug delivery in a localized manner over a prolonged period of time.

### CROSS-REFERENCE TO RELATED APPLICATIONS

The present application is a continuation-in-part of U.S. Ser. No. 10/245,840 filed Sep. 17, 2002, which claims the benefit of U.S. Provisional Application Nos. 60/322,428, filed Sep. 17, 2001 and 60/372,761, filed Apr. 15, 2002; the specifications of each of which are hereby incorporated by reference in their entirety.

### BACKGROUND OF THE INVENTION

Modern surgical methods employ various and numerous devices that are routinely placed within the body and left there for extended periods of time. Such devices include, but are not limited to sutures, stents, surgical screws, prosthetic joints, artificial valves, plates, pacemakers, etc. Such devices have proven useful over time, but some problems associated with implanted surgical devices remain.

For instance, stents, artificial valves, and to some extent even sutures may be associated with restenosis, fibrosis and other proliferative disorders after vascular surgery and inflammation at the site of the surgery, necessitating the administration of pharmaceuticals to prevent or to counteract such undesirable effects of the surgery. In addition, despite many advances that have been made to reduce the exposure of patients to pathogenic microbes during surgery, implantation of surgical devices nonetheless involves introducing into the body a foreign object that has the potential to infect patients with various viruses and/or bacteria. Accordingly, surgical procedures often result in infections to which a patient would not ordinarily be exposed, and which may compromise or negate the effectiveness of implantation therapy. Administration of antibiotics and/or antivirals is therefore a common adjunct to implantation therapy, either for prophylaxis or in response to infection. This often necessitates the systemic delivery of drugs in conjunction with implantation of surgical devices.

However, systemic administration of drugs often leads to undesirable side effects, such as the increased risk of post-operative hemorrhage and impairment of other, healthy bodily functions. Occasionally, surgical implants may be subject to immune response or rejection. Consequently, it is sometimes necessary to abandon surgical implant therapy, or to use immune suppressant drugs in conjunction with certain surgical implants. In particular, there are complications associated with the use of stents that need to be alleviated.

A stent is a generally longitudinal tubular device formed of biocompatible material, preferably a metallic or plastic material. Stents are useful in the treatment of stenosis, strictures or aneurysms in body vessels, such as blood vessels. It is well-known to employ a stent for the treatment of diseases of various body vessels. The device is implanted either as a “permanent stent” within the vessel to reinforce collapsing, partially occluded, weakened or abnormally dilated sections of the vessel or as a “temporary stent” for providing therapeutic treatment to the diseased vessel. Stents are typically employed after angioplasty of a blood vessel to prevent restenosis of the diseased vessel. Stents may be useful in other body vessels, such as the urinary tract and the bile duct.

A typical stent includes an open flexible configuration. The stent configuration allows the stent to be configured in a radially compressed state for intraluminal catheter insertion into an appropriate site. Once properly positioned within the lumen of a damaged vessel, the stent is radially expanded to support and reinforce the vessel. Radial expansion of the stent may be accomplished by an inflatable balloon attached to the catheter, or the stent may be of the self-expanding type that will radially expand once deployed. An example of a suitable stent is disclosed in U.S. Pat. No. 4,733,665, which is incorporated herein by reference in its entirety.

Stents find various uses in surgical procedures. For instance, stents are widely used in angioplasty. Angioplasty involves insertion of a balloon-tipped catheter into an artery at the site of a partially obstructive atherosclerotic lesion. Inflation of the balloon can rupture the intima and media, dramatically dilating the vessel and relieving the obstruction. About 20 to 30% of obstructions reocclude in a few days or weeks, but most can be redilated successfully. Use of stents significantly reduces the reocclusion rate. Repeat angiography one year after angioplasty reveals an apparently normal lumen in about 30% of vessels on which the procedure has been performed.

Angioplasty is an alternative to bypass surgery in a patient with suitable anatomic lesions. The risk is comparable with that of surgery. Mortality is 1 to 3%; myocardial infarction rate is 3 to 5%; emergency bypass for intimal dissection with recurrent obstruction is required in <3%; and the initial success rate is 85 to 93% in experienced hands.

Stents are also used in percutaneous endovascular therapy. Many new treatments for vascular disease (occlusions and aneurysms) avoid open surgery. These treatments may be performed by interventional radiologists, vascular surgeons, or cardiologists. The primary approach is percutaneous translumninal angioplasty (PTA), whereby a small high-pressure balloon is used to open an obstructed vessel. However, because of the high recurrence rate of obstruction, alternative methods may be necessary.

A stent, such as a metallic mesh-like tube, is generally inserted into a vessel at an obstructed site. As stents can be very strong, they tend to keep vessels open much better than balloons alone. Moreover, the recurrence rate of obstruction is reportedly lower when stents are used. Stents work well in larger arteries with high flow, such as iliac and renal vessels. They work less well in smaller arteries, and in vessels in which the occlusions are long. Stents for carotid disease are being studied.

There are at least two known causes of post-operative restenosis—elastic recoil, wherein the vessel contracts due to the natural elasticity of the vessel walls, and neointimal hyperplasia, wherein medial cells proliferate in response to immune system triggers. Stents have proven useful in reducing the incidence and/or severity of post-operative elastic recoil restenosis, as they resist the tendency of blood vessels to restenose after removal of the balloon. Stents have proven less useful for treatment of neointimal hyperplasia, which arises out of a complex immune response to expanding and fracturing the atherosclerotic plaque. In the case of neointimal hyperplasia, the initial expansion and fracture of the atherosclerotic lesion initiates inflammation, which gives rise to a complex cascade of cellular events that activates the immune system, which in turn gives rise to the release of cytokines that stimulate cell multiplication in the smooth muscle layers of the vessel media. This cell stimulation eventually causes the vessel to restenose.

Various approaches to the problem of neointimal hyperplasia have been attempted. Among these approaches are: subsequent stent placement, debulking, repeat angioplasty, and laser treatment. Another recent approach has been to coat the stent with an immunosuppressant or a chemotherapeutic drug. Immunosuppressant drugs, such as rapamycin, target cells in the G1 phase, preventing initiation of DNA synthesis. Chemotherapeutic drugs, such as paclitaxel (Taxol—Bristol-Myers Squibb) and other taxane derivatives, act on cells in the M phase, by preventing deconstruction of microtubules, thereby interrupting cell division. Since many immunosuppressant or chemotherapeutic drugs, as well as potent anti-inflammatory drugs, exert undesirable side effects when administered systemically, coated stents offer an advantage of localized drug delivery that may reduce such side effects. While these approaches present some promise, they also suffer certain limitations, such as the tendency for rapamycin and taxanes to quickly disperse from the stent site, thereby both limiting the drugs' effective duration in proximity to the stent and also risking undesirable local and/or systemic toxic effects.

There is therefore a need for an improved stent that will provide sustained-release of pharmaceutically active compounds, such as chemotherapeutic drugs, at or near the site of stent implantation that alleviates or avoids the problem of rapid depletion of drug from the stent site. There is also a need for an improved drug that may be employed in such a stent.

There is furthermore a need for an improved stent that will provide sustained-release of pharmaceutically active compounds, such as immunosuppressant, chemotherapeutic, and anti-inflammatory drugs, at or near the site of stent implantation that does not suffer the drawbacks of causing systemic toxic effects of the immunosuppressant, chemotherapeutic, and anti-inflammatory drugs. There is also a need for an improved drug that may be employed in such a stent.

### SUMMARY OF THE INVENTION

The subject invention provides medical devices having a coating disposed on at least one surface, wherein the coating includes a polymer matrix and a nucleoside analog or codrug or prodrug thereof (collectively herein “nucleoside analog agent”). In preferred embodiments, the coatings are intended to provide sustained release of an effective amount of the nucleoside analog agent. The subject coatings can be applied to surgical implements such as screws, plates, washers, sutures, prosthesis anchors, tacks, staples, electrical leads, valves, membranes. The devices can be, merely for illustration, catheters, implantable vascular access ports, blood storage bags, blood tubing, central venous catheters, arterial catheters, vascular grafts, intraaortic balloon pumps, heart valves, cardiovascular sutures, artificial hearts, a pacemaker, ventricular assist pumps, extracorporeal devices, blood filters, hemodialysis units, hemoperfusion units, plasmapheresis units, and filters adapted for deployment in a blood vessel.

In certain embodiments, the subject medical device is an intraluminal medical device, e.g., a stent. In a preferred embodiment, the medical device is a vascular stent. In certain instances, particularly where the stent is an expandable stent, the coating is flexible to accommodate compressed and expanded states of the stent.

While exemplary embodiments of the invention will be described with respect to the treatment of restenosis and related complications following percutaneous transluminal coronary angioplasty, it is important to note that the local delivery of nucleoside analog agents may be utilized to treat a wide variety of conditions utilizing any number of medical devices, or to enhance the function and/or life of the device. For example, intraocular lenses, placed to restore vision after cataract surgery is often compromised by the formation of a secondary cataract. The latter is often a result of cellular overgrowth on the lens surface and can be potentially minimized by delivering a nucleoside analog agent with the device. Other medical devices which often fail due to tissue in-growth or accumulation of proteinaceous material in, on and around the device, such as shunts for hydrocephalus, dialysis grafts, colostomy bag attachment devices, ear drainage tubes, leads for pace makers and implantable defibrillators can also benefit from the drug eluting coatings of the present invention.

Devices which serve to improve the structure and function of tissue or organ may also show benefits when combined with the appropriate nucleoside analog agents. Surgical devices, sutures, staples, anastomosis devices, vertebral disks, bone pins, suture anchors, hemostatic barriers, clamps, screws, plates, clips, vascular implants, tissue adhesives and sealants, tissue scaffolds, various types of dressings, bone substitutes, intraluminal devices, and vascular supports could also provide enhanced patient benefit using the subject coatings. Essentially, any type of medical device may be coated in some fashion with nucleoside analog agents.

The subject devices can be used to deliver a wide variety of nucleoside analog agents, such as pyrimidine analogs (fluorouracil, floxuridine, capecitabine, gemcitabine and cytarabine) and purine analogs and related inhibitors (mercaptopurine, thioguanine, pentostatin and 2-chlorodeoxyadenosine (cladribine)) that possess anti-neoplastic/anti-cancer activities.

In certain embodiments, the nucleoside analogs have one or more of the following activities: folate antagonist, DNA damaging agent, DNA synthesis inhibitor, thymidilate synthase inhibitor, DNA polymerase inhibitor and/or DNA repair inhibitor.

In certain preferred embodiments, the nucleoside analog is a halogenated purine or pyrimidine, such as a fluorinated purine or pyrimidine. Even more preferably, it is 5-fluorouracil (“5-FU”) or a prodrug thereof.

In certain preferred embodiments, the nucleoside analog agents are formulated in the polymer matrix as the single pharmaceutical agent. In other embodiments, the nucleoside analog agents can be formulated in combination with, or codruged with, other pharmaceutically active drugs. Such pharmaceutical agents include, merely to illustrate: antiproliferative/antimitotic agents including natural products such as vinca alkaloids (i.e. vinblastine, vincristine, and vinorelbine), paclitaxel, epidipodophyllotoxins (i.e. etoposide, teniposide), antibiotics (dactinomycin (actinomycin D) daunorubicin, doxorubicin and idarubicin), anthracyclines, mitoxantrone, bleomycins, plicamycin (mithramycin) and mitomycin, enzymes (L-asparaginase which systemically metabolizes L-asparagine and deprives cells which do not have the capacity to synthesize their own asparagine); antiplatelet agents; antiproliferative/antimitotic alkylating agents such as nitrogen mustards (mechlorethamine, cyclophosphamide and analogs, melphalan, chlorambucil), ethylenimines and methylmelamines (hexamethylmelamine and thiotepa), alkyl sulfonates-busulfan, nirtosoureas (carmustine (BCNU) and analogs, streptozocin), trazenes-dacarbazinine (DTIC); antiproliferative/antimitotic antimetabolites such as folic acid analogs (methotrexate; platinum coordination complexes (cisplatin, carboplatin), procarbazine, hydroxyurea, mitotane, aminoglutethimide; hormones (i.e., estrogen); anticoagulants (heparin, synthetic heparin salts and other inhibitors of thrombin); fibrinolytic agents (such as tissue plasminogen activator, streptokinase and urokinase), aspirin, dipyridamole, ticlopidine, clopidogrel, abciximab; antimigratory; antisecretory (breveldin); immunosuppressives (cyclosporine, tacrolimus (FK-506), sirolimus (rapamycin), azathioprine, mycophenolate mofetil); corticosteroids angiogenic agents; vascular endothelial growth factor (VEGF), fibroblast growth factor (FGF); angiotensin receptor blocker; nitric oxide donors; anti-sense oligionucleotides and combinations thereof; cell cycle inhibitors, mTOR inhibitors, and growth factor signal transduction kinase inhibitors.

In certain preferred embodiments, the duration of release of an effective amount of the nucleoside analog from the polymer matrix occurs for at least 24 hours, and even more preferably may be for at least 15, 30, 45 or even 60 days. In certain embodiments, the duration of release of an effective amount of the corticosteroid occurs for at least six months.

Appropriate sustained release profiles may be achieved for the release of the nucleoside analog in a number of different ways, and may yield profiles having such characteristics as: a) constant release with time, (b) release rate diminishing with time, c) burst release, and d) pulsed release where some portion of the active material is released suddenly at a certain time. The rate of release, as well as the manner of release, of the nucleoside analog may be varied, for example, by regulating the rate of dissolution, the rate of permeability, the rate of polymer degradation or bioerosion, or the swelling rates, which in turn may be controlled by the pH, moisture and temperature of the environment, chemical properties of the polymeric matrix, such as for example its size, shape and thickness, as well as the size of the polymer matrix pores.

The release rate of the nucleoside analog can also be affected by co-formulation of the nucleoside analog with one or more additives or solvents that affect the solubility and/or rate of diffusion of the nucleoside analog through the polymer matrix, thereby generating a sustained release system.

For instance, nucleoside analogs which are intrinsically soluble can be co-formulated with release-modifying agents that decrease the solubility of the nucleoside analog in the matrix or otherwise slow its release from the polymer matrix. Merely to illustrate, in certain embodiments the polymer matrix encapsulating the polymer has pores or passages that are blocked with one or more additives that have a suitable rate of solubility. The rate of release of the pharmaceutical agent is essentially the rate of solubilization of such additives; the dissolution of the additives makes the polymer matrix more permeable to the pharmaceutical agents, allowing the diffusion of the agents into the surrounding biological fluid. A soluble nucleoside analog can thus be made into a sustained release form by virtue of its mixture with release-modifying agents in the polymer matrix.

In certain embodiments, the subject invention permits the sustained release of a nucleoside analog that is otherwise soluble in water or serum (e.g., having a solubility of at least 1 mg/mL in water at 25C.), by providing a formulation having reduced solubility characteristics. For instance, the nucleoside analog can be provided as a prodrug or codrug that has a logP value at least 0.5 logP units more than the logP value for nucleoside analog, and even more prefereably at least 1, 1.5 or even 2 logP units greater. In certain embodiments, the nucleoside analog is provided as a prodrug or codrug, or otherwise formulated in the polymer coating, to have a solubility in water at 25° C. at least 25 percent less than the solubility of the nucleoside analog, and even more preferably at least 50, 75, 90 or even 95 percent less. In certain embodiments, the nucleoside analog is provided as a prodrug or codrug, or otherwise formulated in the polymer coating, to have a solubility in water at 25° C. less than 0.5 mg/mL, and even more preferably less than 0.1 mg/mL, 0.05 mg/mL or even 0.01 mg/mL.

In other embodiments, the nucleoside analog is insoluble and its rate of release can be increased by addition of one or more additives that increase the rate of release of the drug from the coating.

The choice of polymer may also influence the rate of release of the nucleoside analog. For instance, rate of release of the nucleoside analog can be affected by the pore size of the polymeric matrix pores, or the particular choice of polymer subunits, subsequent chemical modification of the polymer and/or solvent. Diffusion of soluble nucleoside analogs, for example, can be reduced by such manipulation.

In other cases, an otherwise soluble nucleoside analog (such as 5FU) may be rendered as a reduced or low solubility agent through reversible covalent or ionic modification, e.g., as a codrug or prodrug, in order to provide the appropriate sustained release profile. In such embodiments, the codrug or prodrug is preferably relatively insoluble in aqueous media, including physiological fluids, such as blood serum, mucous, peritoneal fluid, limbic fluid, etc.

Likewise, the release profile for an insoluble nucleoside analog can be increased by modification with a hydrophilic group.

To further illustrate, the nucleoside analog can be provided in the form of a codrug or prodrug represented by the general formula A—L—B, in which: A represents a nucleoside analog or prodrug thereof; B represents a moiety which, when linked to A, results in a compound having an optimized solubility for sustained delivery of the nucleoside analog in vivo from the coated device; and L represents a covalent bond or covalent linker linking A and B to form the codrug, wherein the bond or linker is metabolized (such as by hydrolysis) under physiological conditions. When B is a pharmaceutically active moiety, or prodrug thereof, than the resulting covalent molecule is a “codrug”. When B is essentially inert, the moiety is referred to as a “prodrug”.

In other instances, the nucleoside analog is provided as a codrug or prodrug represented by the general formula of A::B, in which: A represents a nucleoside analog or prodrug thereof; B represents a moiety which, when linked to A, results in a compound having an optimized solubility for sustained delivery of the nucleoside analog in vivo from the coated device; and :: represents an ionic bond between A and B that dissociates under physiological conditions.

The nucleoside analog may also be covalently linked to the polymer matrix. The linker is cleaved under physiological conditions to release the pharmaceutically active form of the drug. In certain embodiments, the linkage is hydrolyzed in bodily fluid. In other embodiments, the linkage is enzymatically cleaved. The drug or prodrug is released into the environment upon cleavage of the covalent bond either by hydrolysis or by enzymatic cleavage once the linkage is exposed to the surrounding biological fluid. In certain embodiments, the polymer matrix is bioerodible, and the rate of release of the drug or prodrug is essentially the same as the rate of bioerosion. Once the polymer is degraded, the bond that links the pharmaceutical agents to the polymer is rapidly cleaved and the drugs are released. In other embodiments, the polymer matrix is non-bioerodible or, alternatively, bioerodible at such a rate of bioerosion that the rate of the cleavage of the covalent bond is essentially the rate of release of the drug or prodrug into the surrounding biological fluid.

Examples of linkages which can be used include one or more hydrolysable groups selected from the group consisting of an ester, an amide, a carbamate, a carbonate, a cyclic ketal, a thioester, a thioamide, a thiocarbamate, a thiocarbonate, a xanthate and a phosphate ester.

Alternatively, the nucleoside analog is not covalently linked to the polymer, but its rate of release is nevertheless controlled by the rate of biodegradation or bioerosion of the polymer matrix.

Exemplary bioerodible polymer matrices can be formed polyanhydride, polylactic acid, polyglycolic acid, polyorthoester, polyalkylcyanoacrylate, and derivatives and copolymers thereof.

In certain embodiments, the polymer matrix is non-bioerodible, while in other embodiments it is bioerodible. In certain embodiments, the polymer matrix is a mixture of non-bioerodible and bioerodible materials. Exemplary non-bioerodible polymer matrices can be formed from polyurethane, polysilicone, poly(ethylene-co-vinyl acetate), polyvinyl alcohol, and derivatives and copolymers thereof.

In certain embodiments, the polymer matrix is non-bioerodible, but is impregnated with water-soluble or bioerodible components that control the matrix pore size. As the water soluble components leach out into the surrounding physiological fluid, the matrix pores enlarge, creating a larger surface area which allows the drug or prodrug to be released into the fluid.

In certain embodiments, the polymer matrix is chosen so as reduce interaction between the prodrug in the matrix and proteinaceous components in surrounding bathing fluid, e.g., by forming a matrix have physical (pore size, etc) and/or chemical (ionized groups, hydrophobicity, etc) characteristics which exclude proteins from the inner matrix, e.g., exclude proteins of greater than 100 kDa, and even more preferably exclude proteins greater in size than 50 kDa, 25 kDa, 10 kDa or even 5 kDa.

In certain embodiments, the polymer matrix is essentially non-release rate limiting with respect to the rate of release of the nucleoside analog from the matrix.

In other embodiments, the subject polymer matrices influence the rate of release of the nucleoside analog. For instance, the matrices can be derived to have charge or hydrophobicity characteristics which favor sequestration of a derivative of the nucleoside analog, such as codrug or prodrug forms, over the released active nucleoside analog. Likewise, the polymer matrix can influence the pH-dependency of the hydrolysis or other reaction for converting a prodrug or codrug form of a nucleoside analog into an active nucleoside analog moiety, or create a microenvironment having a pH different than the bathing bodily fluid, such that hydrolysis and/or solubility of the codrug or prodrug is different within the matrix than in the surrounding fluids. In such a manner, the polymer can influence the rate of release, and the rate of hydrolysis of the codrug or prodrug, by differential electronic, hydrophobic or chemical interactions with the derivative.

In certain embodiments, the polymer is chosen based on the solubility of the nucleoside analog in the polymer of hydrated polymer.

In certain embodiments, the weight of the coating attributable to the nucleoside analog (or its codrug or prodrug forms) is in the range of about 0.05 mg to about 50 mg of prodrug per cm² of the surface coated with said polymer matrix, and even more preferably 5 to 25 mg/cm².

In certain embodiments, the coating has a thickness is in the range of 5 micrometers to 100 micrometers.

In certain embodiments, the nucleoside analog (or codrug or prodrug thereof) is present in the coating in an amount between 5% and 70% by weight of the coating, and even more preferably 25 to 50% by weight.

Another embodiment according to the present invention is advantageously a solid device of a shape and form suitable for implantation. The polymer is rigid and comprises part or whole of an implantable medical device, such as a screw, stent, prosthetic joint, etc. Alternatively, the polymer is pliant and is formed in shape of sutures.

In embodiments according to the present invention wherein the device comprises a substrate and a coating on the substrate, such as a screw, stent, pacemaker, prosthetic joint, etc., the device is used in substantially the manner of the corresponding prior art surgical implement. For instance, a device according to the present invention that comprises a screw coated with a composition comprising a nucleoside analog, such as 5FU or a codrug or prodrug thereof, suspended or dispersed in a polymer, is screwed into a bone in the same manner as a prior art screw. The screw according to the present invention then releases drug, in a sustained time-wise fashion, thereby conferring therapeutic benefits to the tissue surrounding the device, such as muscle, bone, blood, etc.

Yet another aspect of the invention provides a method for treating an intraluminal tissue of a patient. In general, the method comprising the steps of:

(a) providing a stent having an interior surface and an exterior surface, said stent having a coating on at least a part of the interior surface, the exterior surface, or both; said coating comprising a nucleoside analog formulation dissolved or dispersed in a biologically-tolerated polymer;

(b) positioning the stent at an appropriate intraluminal tissue site; and

(c) deploying the stent.

In such embodiments, the drug combinations and delivery devices of the present invention may be utilized to effectively prevent and treat vascular disease, and in particular, vascular disease caused by injury.

Another aspect of the invention relates to a coating composition for use in delivering a medicament from the surface of a medical device positioned in vivo. The composition comprises a polymer matrix and a nucleoside analog as described above. The coating composition can be provided in liquid or suspension form for application to the surface of a medical device by spraying and/or dipping the device in the composition. In other embodiments, the coating composition is provided in powdered form and, upon addition of a solvent, can reconstitute a liquid or suspension form for application to the surface of a medical device by spraying and/or dipping the device in the composition.

Additional advantages of the present invention will become readily apparent to those skilled in the art from the following detailed description, wherein only a preferred embodiment of the invention is shown and described by way of illustration of the best mode contemplated for carrying out the invention. As will be realized, the present invention is capable of other and different embodiments, and its several details are capable of modifications in various respects, all without departing from the scope of the present invention. Accordingly, the drawings and description are to be regarded as illustrative in nature, and not as restrictive.

### BRIEF DESCRIPTION OF THE DRAWINGS

The following description of the embodiment of the present invention can be better understood when read in conjunction with the following drawings, in which like reference numerals are employed throughout to designate similar features, wherein:

FIG. 1 is a side plan view of a non-deployed stent according to the present invention.

FIG. 2 is a side plan view of a deployed stent according to the present invention.

FIG. 3 is a release profile of 5-Fluroruracil (5FU) and triamcinolone acetonide (TA) from coated inserts.

FIG. 4 is a release profile of 5-flurouracil (5FU) and triamcinolone acetonide (TA) from coated inserts.

FIG. 5 illustrates the release pattern in vitro for a high dose coated stent.

FIG. 6 shows the comparative drug release profiles between explanted stents and non-implanted stents.

FIG. 7 shows the release rate from stents that were coated with a mixture of TA and 5FU in a mole-ratio of 1 to 1 without chemical linkage.

FIGS. 8A and 8B are graphs showing the effect of gamma irradiation and plasma treatment on drug release. Group B: with plasma treatment, with gamma irradiation. Group C: no plasma treatment, with gamma irradiation. Group D: with plasma treatment, no gamma irradiation. Group F: no plasma, no gamma irradiation.

FIG. 9 is a release profile of Cyclosporin A (CsA) from an implant coated with a silicone-CsA matrix.

### DETAILED DESCRIPTION OF THE INVENTION

I. Definitions

The term “pharmaceutically active moiety” means any physiologically or pharmacologically active chemical entity that produces a desired local or systemic effect in a treated animal, e.g., in a human patient, and preferably with an ED₅₀ of 1 mM or less, and more preferably less than 1 μM. This is in contrast to a chemical entity that is inert or merely pyrogenic.

The term “biological fluid” means any aqueous solution found naturally in the body of a living animal, including but not limited to, serum, lymph, synovial fluid, any exudates, amniotic fluid, saliva, urine or cerebral spinal fluid.

“LogP” refers to the logarithm of P (Partition Coefficient). P is a measure of how well a substance partitions between a lipid (oil) and water. P itself is a constant. It is defined as the ratio of concentration of compound in aqueous phase to the concentration of compound in an immiscible solvent, as the neutral molecule.

Partition Coefficient, P=[Organic]/[Aqueous] where []=concentration

LogP=log₁₀ (Partition Coefficient)=log₁₀P

A LogP value of 1 means that the concentration of the compound is ten times greater in the organic phase than in the aqueous phase. The increase in a logP value of 1 indicates a ten-fold increase in the concentration of the compound in the organic phase as compared to the aqueous phase.

A “patient” or “subject” can mean either human or non-human animal.

A “purine or pyrimidine anti-neoplastic agent” is a chemical compound with an anti-neoplastic activity, the structure of which includes a purine or a pyrimidine moiety. A purine or pyrimidine anti-neoplastic agent may be, but does not have to be, a nucleoside or a nucleotide.

In the context of referring to a codrug, the term “residue” means that part of a codrug that is structurally derived from a pharmaceutically active moiety or its prodrug. Where the codrug includes covalently linked pharmaceutically active moieties, at least one of the groups of the residue will be varied (relative to the pharmaceutically active moiety or its prodrug) to accommodate the covalent linker. For instance, where the codrug includes an amino functional group, the residue may form an amide (—NH—CO—) bond with another residue of the codrug. In this sense, the term “residue” as used herein is analogous to the sense of the word “residue” as used in peptide and protein chemistry to refer to a residue of an amino acid in a peptide.

A “prodrug” is a compound that may not be pharmacologically active, but is at least less active then a metabolite thereof. That is, the ED₅₀ for a biological activity of a prodrug is usually greater than for one or more of its metabolites. However, when activated in vivo by metabolic (such as enzymatic) or non-enzymatic hydrolytic cleavage, the prodrug is converted to a pharmaceutically active moiety. Prodrugs are typically formed by chemical modification of a pharmaceutically active moiety.

The terms “linker” and “linkage”, which are used interchangeably herein, refers to a direct bond or group of atoms incorporating and connecting the functional groups of two or more discrete and otherwise separate pharmaceutically active moieties, and which is metabolized under physiological conditions to generate the two or more pharmaceutically active moieties or their prodrugs. Preferably, the linker moiety is typically a substantially linear moiety, and includes no more than 25 atoms, and even more preferably less than 10 atoms. Preferred linkers are ones which, when metabolized, generate the pharmaceutically active moieties (or their prodrugs) as discrete and separate chemical entities, and if any byproducts also result, such byproducts are generally inert at the dosing concentration of the codrug.

“Physiological conditions” describe the in vivo conditions to which the prodrug or codrug is subjected. Physiological conditions include the acidic and basic environments of body cavities and organs, biological fluids, and intracellular or extracellular millieu.

The term “ED₅₀” means the dose of a drug which produces 50% of its maximum response or effect. Alternatively, the dose which produces a pre-determined response in 50% of test subjects or preparations.

II. Exemplary Embodiments

The present invention provides a system comprising a coated medical device, the coating of which is suitable for sustained release of nucleoside analogs in the locality of the implanted device. Exemplary embodiments are described using an intraluminal medical device, particularly a stent, but the inventive system is also readily applicable to and advantageous in other forms of medical devices.

Once administered, the system remains in the body and serves as a continuous source of the corticosteroid to the affected area. The system according to the present invention permits prolonged release of corticosteroid(s) over a specific period of days, weeks, months (e.g., about 3 months to about 6 months) or years (e.g., about 1 year to about 20 years, such as from about 5 years to about 10 years) until the drug reservoir is used up.

In certain embodiments, the present invention provides an intraluminal medical device for implantation into a lumen of a blood vessel, in particular adjacent an intraluminal lesion such as an atherosclerotic lesion, for maintaining patency of the vessel. In particular embodiments, the present invention provides an elongate radially expandable tubular stent having an interior luminal surface and an opposite exterior surface extending along a longitudinal stent axis, the stent having a coating on at least a portion of the interior or exterior surface thereof. The local delivery of nucleoside analogs from a stent has the following advantages; namely, the prevention of vessel recoil and remodeling through the scaffolding action of the stent and the prevention of multiple components of neointimal hyperplasia or restenosis as well as a reduction in inflammation and thrombosis. This local administration of nucleoside analogs to stented coronary arteries may also have additional therapeutic benefit. For example, higher tissue concentrations of the nucleoside analogs may be achieved utilizing local delivery, rather than systemic administration. In addition, reduced systemic toxicity may be achieved utilizing local delivery rather than systemic administration while maintaining higher tissue concentrations. Also in utilizing local delivery from a stent rather than systemic administration, a single procedure may suffice with better patient compliance. In case of combination therapy, an additional benefit may be to reduce the dose of each of the nucleoside analogs or other therapeutic drugs, agents or compounds, thereby limiting their toxicity, while still achieving a reduction in restenosis, inflammation and thrombosis. Local stent-based therapy is therefore a means of improving the therapeutic ratio (efficacy/toxicity) of anti-restenosis, anti-inflammatory, anti-proliferative, anti-thrombotic drugs, agents or compounds.

There are a multiplicity of different stents that may be utilized following percutaneous transluminal coronary angioplasty. Although any number of stents may be utilized in accordance with the present invention, for simplicity, a limited number of stents will be described in exemplary embodiments of the present invention. The skilled artisan will recognize that any number of stents may be utilized in connection with the present invention.

In addition, as stated above, other medical devices may be utilized. In other embodiments according to the present invention, the polymer in which a sustained release corticosteroid formulation is suspended or dispersed is coated onto a surgical implement such as surgical tubing (such as colostomy, peritoneal lavage, catheter, and intravenous tubing). In still further embodiments according to the present invention, the device is an intravenous needle having the polymer and a corticosteroid (or codrug or prodrug thereof) coated thereon.

A stent is commonly used as a tubular structure left inside the lumen of a duct to relieve an obstruction. Commonly, stents are inserted into the lumen in a non-expanded form and are then expanded autonomously, or with the aid of a second device in situ. A typical method of expansion occurs through the use of a catheter-mounted angioplasty balloon which is inflated within the stenosed vessel or body passageway in order to shear and disrupt the obstructions associated with the wall components of the vessel and to obtain an enlarged lumen.

The stents of the present invention may be fabricated utilizing any number of methods. For example, the stent may be fabricated from a hollow or formed stainless steel tube that may be machined using lasers, electric discharge milling, chemical etching or other means. The stent is inserted into the body and placed at the desired site in an unexpanded form. In one exemplary embodiment, expansion may be effected in a blood vessel by a balloon catheter, where the final diameter of the stent is a function of the diameter of the balloon catheter used.

It should be appreciated that a stent in accordance with the present invention may be embodied in a shape-memory material, including, for example, an appropriate alloy of nickel and titanium or stainless steel.

Structures formed from stainless steel may be made self-expanding by configuring the stainless steel in a predetermined manner, for example, by twisting it into a braided configuration. In this embodiment after the stent has been formed it may be compressed so as to occupy a space sufficiently small as to permit its insertion in a blood vessel or other tissue by insertion means, wherein the insertion means include a suitable catheter, or flexible rod.

On emerging from the catheter, the stent may be configured to expand into the desired configuration where the expansion is automatic or triggered by a change in pressure, temperature or electrical stimulation.

Regardless of the design of the stent, it is preferable to have the sustained release nucleoside analog formulation applied with enough specificity and a sufficient concentration to provide an effective dosage in the lesion area. In this regard, the “reservoir size” in the coating is preferably sized to adequately apply the nucleoside analog formulation at the desired location and in the desired amount and for a sustained period of time.

In an alternate exemplary embodiment, the entire inner and outer surface of the stent may be coated with drug/drug combinations in therapeutic dosage amounts. It is, however, important to note that the coating techniques may vary depending on the drug combinations. Also, the coating techniques may vary depending on the material comprising the stent or other intraluminal medical device.

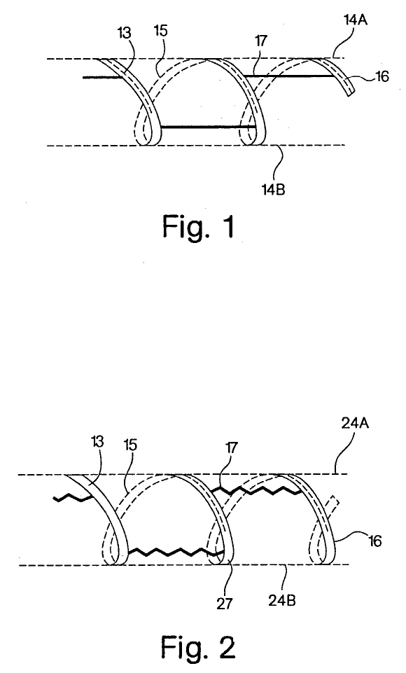

An embodiment of an intraluminal device (stent) according to the present invention is depicted in FIGS. 1 and 2.

FIG. 1 shows a side plan view of a preferred elongate radially expandable tubular stent 13 having a surface coated with a sustained release drug delivery system in a non-deployed state. As shown in FIG. 1, the stent 13 has its radially outer boundaries 14A, 14B at a non-deployed state. The interior luminal surface 15, the exterior surface 16, or an entire surface of the stent 13 may be coated with a sustained release drug delivery system or comprise a sustained release drug delivery system. The interior luminal surface 15 is to contact a body fluid, such as blood in a vascular stenting procedure, while the exterior surface 16 is to contact tissue when the stent 13 is deployed to support and enlarge the biological vessel or duct.

In an alternate embodiment, an optional reinforcing wire 17 that connects two or more of the adjacent members or loops of the stent structure 13 is used to lock-in and/or maintain the stent at its expanded state when a stent is deployed. This reinforcing wire 17 may be made of a Nitinol or other high-strength material. A Nitinol device is well known to have a preshape and a transition temperature for said Nitinol device to revert to its preshape. One method for treating an intraluminal tissue of a patient using a surface coated stent 13 of the present invention comprises collapsing the radially expandable tubular stent and retracting the collapsed stent from a body of a patient. The operation for collapsing a radially expandable tubular stent may be accomplished by elevating the temperature so that the reinforcing wire 17 is reversed to its straightened state or other appropriate state to cause the stent 13 to collapse for removing said stent from the body of a patient.

FIG. 2 shows an overall view of an elongate radially expandable tubular stent 13 having a sustained release drug delivery system coated stent surface at a deployed state. As shown in FIG. 2, the stent 13 has its radially outer boundaries 24A, 24B at a deployed state. The interior luminal surface 14, the exterior surface 16, or an entire surface of the stent 13 may be coated or may comprise the sustained release drug delivery system. The interior luminal surface 15 is to contact a body fluid, such as blood in a vascular stenting procedure, while the exterior surface 16 is to contact tissue when the stent 13 is deployed to support and enlarge the biological vessel. The reinforcing wire 17 may be used to maintain the expanded stent at its expanded state as a permanent stent or as a temporary stent. In the case of the surface coated stent 13 functioning as a temporary stent, the reinforcing wire 17 may have the capability to cause collapsing of the expanded stent.

The deployment of a stent can be accomplished by a balloon on a delivery catheter or by self-expanding after a pre-stressed stent is released from a delivery catheter. Delivery catheters and methods for deployment of stents are well known to one who is skilled in the art. The expandable stent 13 may be a self-expandable stent, a balloon-expandable stent, or an expandable-retractable stent. The expandable stent may be made of memory coil, mesh material, and the like.

In one embodiment, an intraluminal medical device comprises an elongate radially expandable tubular stent having an interior luminal surface and an opposite exterior surface extending along a longitudinal stent axis. The stent may include a permanent implantable stent, an implantable grafted stent, or a temporary stent, wherein the temporary stent is defined as a stent that is expandable inside a vessel and is thereafter retractable from the vessel. The stent configuration may comprise a coil stent, a memory coil stent, a Nitinol stent, a mesh stent, a scaffold stent, a sleeve stent, a permeable stent, a stent having a temperature sensor, a porous stent, and the like. The stent may be deployed according to conventional methodology, such as by an inflatable balloon catheter, by a self-deployment mechanism (after release from a catheter), or by other appropriate means. The elongate radially expandable tubular stent may be a grafted stent, wherein the grafted stent is a composite device having a stent inside or outside of a graft. The graft may be a vascular graft, such as an ePTFE graft, a biological graft, or a woven graft. As appropriate, the subject sustained release nucleoside analog formulations (e.g., in monomeric, prodrug or codrug form) may be incorporated into the grafted material.

In one embodiment according to the present invention, the exterior surface of the expandable tubular stent of the intraluminal medical device of the present invention comprises a coating according to the present invention. The exterior surface of a stent having a coating is the tissue-contacting surface and is biocompatible. The “sustained release drug delivery system coated surface” is synonymous with “coated surface”, which surface is coated, covered or impregnated with sustained release drug delivery system according to the present invention.

In an alternate embodiment, the interior luminal surface or entire surface (i.e., both interior and exterior surfaces) of the elongate radially expandable tubular stent of the intraluminal medical device of the present invention has the coated surface. The interior luminal surface having the inventive sustained release drug delivery system coating is also the fluid contacting surface, and is biocompatible and blood compatible.

U.S. Pat. Nos. 5,773,019, 6,001,386, and 6,051,576 disclose implantable controlled-release devices and drugs and are incorporated in their entireties herein by reference. The inventive process for making a surface coated stent includes deposition onto the stent of a coating by, for example, dip coating or spray coating. In the case of coating one side of the stent, only the surface to be coated is exposed to the dip or spray. The treated surface may be all or part of an interior luminal surface, an exterior surface, or both interior and exterior surfaces of the intraluminal medical device. The stent may be made of a porous material to enhance deposition or coating into a plurality of micropores on or in the applicable stent surface, wherein the microporous size is preferably about 100 microns or less.

Problems associated with treating restinosis and neointimal hyperplasia can be addressed by the choice of pharmaceutical agent used to coat the stent. In certain preferred embodiments of the present invention, the chosen pharmaceutical agent is a nucleoside analog having anti-neoplastic activity, or a prodrug thereof.

Where the nucleoside analog is provided as a codrug, the second moiety can be the same or different chemical species, and can be formed, as desired, in equimolar or non-equimolar concentrations to provide optimal treatment based on the relative activities and other pharmaco-kinetic properties of the compounds. The drug combination, particularly where codrug formulations are used, may itself be advantageously relatively insoluble in physiologic fluids, such as blood and blood plasma, and has the property of regenerating any or all of the pharmaceutically active compounds when dissolved in physiologic fluids. In other words, the codrug is converted into the constituent pharmaceutically active compounds upon dissolution. The low-solubility of the pharmaceutical agent thus insures persistence of the agent in the vicinity of an intraluminal lesion. The conversion of the low-solubility pharmaceutical agent into the constituent pharmaceutically active compounds insures a steady, controlled, dose of the pharmaceutically active compounds near the site of the lesion to be treated.

Prodrugs and codrugs useful in the present inventions include nucleoside analogs, such as pyrimidine or purine anti-neoplastic agents that have anticancer/anti-proliferative activities. Exemplary nucleoside analogs for use are halogenated purine or pyrimidine analogs, and preferably fluorinated analogs, such as 5-fluorouracil (5FU).

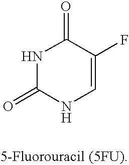

Examples of a suitable second pharmaceutically active compound include immune response modifiers such as cyclosporin A and FK 506, corticosteroids such as dexamethasone, fluocinolone acetonide and triamcinolone acetonide, angiostatic steroids such as trihydroxy steroids, antibiotics including ciprofloxacin, differentiation modulators such as retinoids (e.g., trans-retinoic acid, cis-retinoic acid and analogues), and non-steroidal anti-inflammatory agents such as naproxen, diclofenac, indomethacin and flurbiprofen.

In certain preferred embodiments according to the present invention, the second pharmaceutically active compound is selected from fluocinolone acetonide, triamcinolone acetonide, diclofenac, and naproxen.

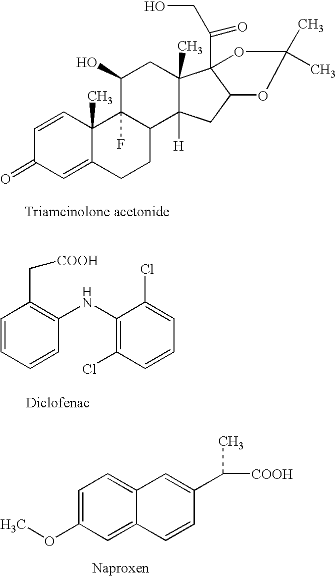

In the case of prodrugs and codrugs, the various moieties can be covalently bonded, connected through a linker, ionically combined, or combined as a mixture.

In some embodiments according to the present invention, the first and second moieties are covalently bonded directly to one another. Where the first and second moieties are directly bonded to one another by a covalent bond, the bond may be formed by forming a suitable covalent linkage through an active group on each compound. For instance, an acid group on one moiety may be condensed with an amine, an acid or an alcohol on the second moiety to form the corresponding amide, anhydride or ester, respectively.

In addition to carboxylic acid groups, amine groups, and hydroxyl groups, other suitable active groups for forming linkages between the two or more moieties include sulfonyl groups, sulfhydryl groups, and the haloic acid and acid anhydride derivatives of carboxylic acids.

In other embodiments, the moieties in the codrug or prodrug embodiments may be covalently linked to one another through an intermediate linker. The linker advantageously possesses two active groups, one of which is complementary to an active group on the nucleoside analog and the other of which is complementary to an active group on the other moiety. For example, where each compound possesses free hydroxyl groups, the linker may suitably be a diacid, which will react with both compounds to form a diether linkage between the two residues. In addition to carboxylic acid groups, amine groups, and hydroxyl groups, other suitable active groups for forming linkages between nucleoside analogs and other compounds include sulfonyl groups, sulfhydryl groups, and the haloic acid and acid anhydride derivatives of carboxylic acids.

In yet another embodiment, the nucleoside analogs may be covalently linked to the polymer matrix, either directly or through an intermediate linker. The characteristics of a desirable linker are analogous to the linker that bonds two moieties to form a prodrug or codrug.

Suitable linkers are set forth in Table 1 below.

TABLE 1

| First Pharmaceutically | Second Pharmaceutically | |
|---|---|---|
| Active Compound Active | Active Compound Active | |
| Group | Group | Suitable Linker |
| Amine | Amine | Diacid |
| Amine | Hydroxy | Diacid |
| Hydroxy | Amine | Diacid |
| Hydroxy | Hydroxy | Diacid |
| Acid | Acid | Diamine |
| Acid | Hydroxy | Amino acid, |
| | | hydroxyalkyl acid, |
| | | sulfhydrylalkyl acid |
| Acid | Amine | Amino acid, |
| | | hydroxyalkyl acid, |
| | | sulfhydrylalkyl acid |

Suitable diacid linkers include oxalic, malonic, succinic, glutaric, adipic, pimelic, suberic, azelaic, sebacic, maleic, fumaric, tartaric, phthalic, isophthalic, and terephthalic acids. While diacids are named, the skilled artisan will recognize that in certain circumstances the corresponding acid halides or acid anhydrides (either unilateral or bilateral) are preferred as linker reagents. A preferred anhydride is succinic anhydride. Another preferred anhydride is maleic anhydride. Other anhydrides and/or acid halides may be employed by the skilled artisan to good effect.

Suitable amino acids include γ-butyric acid, 2-aminoacetic acid, 3-aminopropanoic acid, 4-aminobutanoic acid, 5-aminopentanoic acid, 6-aminohexanoic acid, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Again, the acid group of the suitable amino acids may be converted to the anhydride or acid halide form prior to their use as linker groups.

Suitable diamines include 1,2-diaminoethane, 1,3-diaminopropane, 1,4-diaminobutane, 1,5-diaminopentane, 1,6-diaminohexane.

Suitable aminoalcohols include 2-hydroxy-1-aminoethane, 3-hydroxy-1-aminoethane, 4-hydroxy-1-aminobutane, 5-hydroxy-1-aminopentane, 6-hydroxy-1-aminohexane.

Suitable hydroxyalkyl acids include 2-hydroxyacetic acid, 3-hydroxypropanoic acid, 4-hydroxybutanoic acid, 5-hydroxypentanoic acid, 5-hydroxyhexanoic acid.

The person having skill in the art will recognize that by selecting first and second pharmaceutical moieties (and optionally third, etc., pharmaceutical moieties) having suitable active groups, and by matching them to suitable linkers, a broad palette of inventive compounds may be prepared within the scope of the present invention.

Exemplary preferred codrugs according to the present invention include 5FU covalently bonded to fluocinolone acetonide, 5FU covalently bonded to diclofenac, and 5FU covalently bonded to naproxen. Illustrative examples include the following:

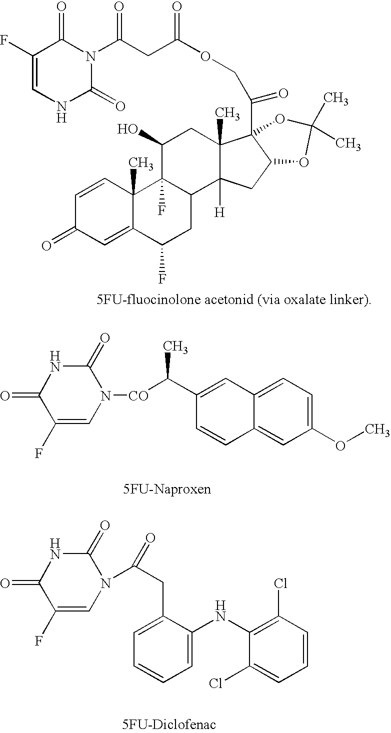

In other embodiments, the nucleoside analog may be combined with other agents to form a salt prodrug or codrug.

In still other embodiments, the nucleoside analog can be combined with other pharmaceutically active compounds as a mixture.

Prodrugs and codrugs of nucleoside analogs, as described herein, will generally be preferred to be slowly dissolved in physiologic fluids, but relatively quickly dissociated to release the nucleoside analog and other actives upon dissolution in physiologic fluids. In some embodiments the dissolution rate of the inventive compounds is in the range of about 0.001 μg/day to about 10 μg/day, e.g., two or three days post-implant. In certain embodiments, the inventive compounds have dissolution rates in the range of about 0.01 to about 1 μg/day. In particular embodiments, the inventive compounds have dissolution rates of about 0.1 μg/day.

In some embodiments according to the present invention, the nucleoside analog (or its prodrug or codrug) is covalently bound to the polymer vehicle. In certain embodiments, the polymer is non-bioerodible or only slightly bioerodible but is permeable to both water and the nucleoside analog. The bond is hydrolysable by the exposure to the physiological fluid. The water from the physiological fluid will permeate the polymer matrix, causing the bond that binds the drug to the polymer to be cleaved. The drug diffuses through the polymer into the surrounding fluid. The rate-limiting step is the permeability of the water into the polymer.

In certain embodiments, the polymer is bioerodible, and the rate of bioerosion is rate-limiting for the release of the nucleoside analog or its prodrug or codrug. For instance, the polymer can include covalent bonds that are hydrolyzed as the polymer matrix is biodegraded. The rate of drug is controlled by the rate of the polymer bioerosion or biodegradation, followed by the rate of hydrolysis that releases the drug from the polymer. Alternatively, the polymer is completely hydrolyzed so that the bound drug is converted into a soluble drug which is free of any residual chemical moiety derived from the polymer to which the drug was bound. The rate of drug release is controlled by the rate of polymer hydrolysis.

Another means for controlling the rate of release of the nucleoside analog relies on including one or more additives in the polymer matrix that increase or decrease the rate of release of the agent from the polymer matrix to optimize its release into the surrounding biological fluid. Such additives may bind to the polymer, changing the microenvironment of the polymer matrix and altering the binding of the drugs to the polymer. For example, an additive may bind to and neutralize the ionic charges of the polymer, resulting in weaker ionic binding of drug to the polymer. Examples of such additives are simple salt-forming ions, detergents, fatty acids and derivatives thereof, amphiphilic compounds such as modified oligosaccharides and polysaccharides or acyl or aromatic derivatives with sufficient hydrophilic moieties, which binds to some or all of ionic moieties and/or hydrogen-bonding moieties of the polymer matrix, and which comprise hydrophobic portions that renders microenvironment more hydrophobic.

Alternatively, such additives may bind to one or more drugs and change the affinity of the drug or prodrug to the polymer, and consequently changing the rate of release of the drugs from the polymer. Examples of such additives are monosaccharides, disaccharides, oligosaccharides, polysaccharides such as cyclodextrin, dextran, carrageenan, and sugar alcohols, short-chained polymers such as polyethylene glycol, polyvinyl pyrrolidone, poly detergents, amphiphilic compounds, polyanionic or polycationic compounds, biological macromolecules such as polypeptides and nucleoic acids that may form complexes with the drug or the prodrug.

In other embodiments, the additives may reduce the solubility of a nucleoside analog (or its prodrug or codrug) in the biological fluid surrounding the device, by interacting with the drug or the prodrug in a non-ionic and non-covalent manner, such as by forming hydrogen-bonds or hydrophobic bonds.

The additives may also change the solubility of the nucleoside analogs by dissolving or forming micellar units and by stabilizing emulsions that are more readily dispersed into the biological fluid. Linear alkyl and aromatic detergents and other biphasic compounds may be used for this purpose.

In yet other embodiments of this invention, one or more additives may be used to impregnate the pores of the polymer matrix. The polymer is non-bioerodible or only slightly bioerodible, but its pores are impregnated with additives that are bioerodible or water-soluble. The contact with physiological fluid that surrounds polymer matrix will degrade or dissolve the bioerodible or water-soluble additives and enlarge the pore size of the matrix and increasing the surface area exposed to the biological fluid, thereby exposing the drug or prodrug to the environment. This will allow the drug or prodrug to diffuse out of the matrix more readily. Such additives may be any physiologically inactive compounds which dissolve or degrade over an extended period of time. For example, monosaccharides, disaccharides, oligosaccharides or sugar alcohols, such as xylose, fructose, glucose, sucrose, lactose, maltose, cellobiose, trehalose, arabinose, sorbitol, mannitol, dextran, alginates, chitosan, pectin, hyaluronic acid and cyclodextrin and the derivatives thereof with suitable solubility profiles may be used. Other pharmaceutically inactive compounds that are routinely used as excipients may be adapted for use in the present invention as additives.

Further exemplary materials that may be added to the polymer matrix include hydrophilic polymers, such as polyethylene oxide, polyvinyl pyrrolidone, polyethylene glycol, carboxylmethyl cellulose, hydroxymethyl cellulose and combination thereof to an aliphatic polyester coating to modify the release profile. Appropriate relative amounts can be determined by monitoring the in vitro and/or in vivo release profiles for the therapeutic agents. One or more of these materials can be combined to achieve a desired solubility profile.

Thus, the selection of additives and/or polymer for any given nucleoside analog (or prodrug or codrug thereof) can be used to optimize its release profile by effecting the ability of the drug to partition out of the polymer, through the rate of biodegradation of the polymer, or both.

Various embodiments of this invention comprise polymers with varied physical characteristics. In some embodiments according to the invention, the system comprises a polymer that is relatively rigid. In other embodiments, the system comprises a polymer that is soft and malleable. In still other embodiments, the system includes a polymer that has an adhesive character. Hardness, elasticity, adhesive, and other characteristics of the polymer may be varied as necessary.

Any number of bioerodible or non-erodible polymers may be utilized in conjunction with the drug or drug combination. Polymers may be advantageously selected from among those which reduce the rate of diffusion of the drug or drug combination. Polymers that can be used for coatings in this application can be absorbable or non-absorbable and must be biocompatible to minimize irritation to the vessel wall. The polymer may be either biostable or bioabsorbable depending on the desired rate of release or the desired degree of polymer stability, but a bioabsorbable polymer may be preferred since, unlike biostable polymer, it will not be present long after implantation to cause any adverse, chronic local response.

In some embodiments according to the present invention, the polymer coating is permeable to water in the surrounding tissue, e.g. in blood plasma. In such cases, water solution may permeate the polymer, thereby contacting the drug. The rate of dissolution may be governed by a complex set of variables, such as the polymer's permeability, the solubility of the nucleoside analog agent, the pH, ionic strength, and protein composition, etc. of the physiologic fluid. In certain embodiments, however the permeability may be adjusted so that the rate of dissolution is governed primarily, or in some cases practically entirely, by the solubility of the nucleoside analog in the ambient liquid phase. n still other embodiments the nucleoside agent may have a high solubility in the surrounding fluid. In such cases the matrix permeability may be adjusted so that the rate of dissolution is governed primarily, or in some cases practically entirely, by the permeability of the polymer.

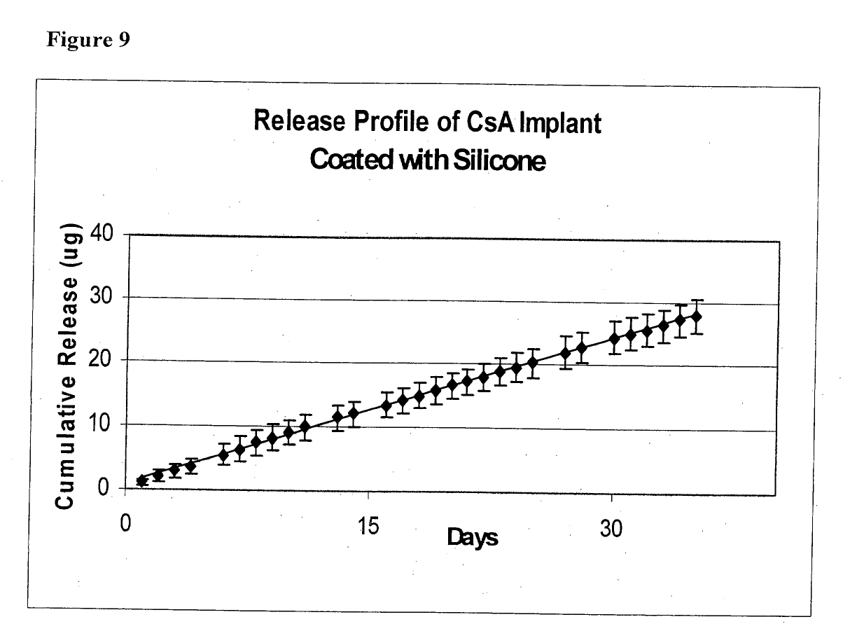

The rate of efflux of drug from the polymer can be affected by such parameters (in the choice of polymer and/or additive) as hydropobic interactions between the drug and polymer and/or additive, ionic or other electrostatic interactions between the drug and polymer and/or additive, hydrogen-bonding between the drug and polymer and/or additive, and pore size of the polymer matrix. FIG. 9 illustrates the effect that electrostatic interaction between the drug and polymer can have on the rate of release. That figure shows the rate of release of cyclosporin A from a silicon matrix. In contrast, the rate of release of flucinolone acetonide, a much smaller molecule, was much lower (below detection limits) presumably due to electrostatic interaction with the silicone matrix.

Suitable bioerodible and bioabsorbable polymers that could be used include polymers selected from the group consisting of aliphatic polyesters, poly(amino acids), copoly(ether-esters), polyalkylenes oxalates, polyamides, poly(iminocarbonates), polyorthoesters, polyoxaesters, polyamidoesters, polyoxaesters containing amido groups, poly(anhydrides), polyphosphazenes, biomolecules and blends thereof. For the purpose of this invention aliphatic polyesters include homopolymers and copolymers of lactide (which includes lactic acid d-,l- and meso lactide), E-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one and polymer blends thereof. Poly(iminocarbonate) for the purpose of this invention include as described by Kemnitzer and Kohn, in the Handbook of Biodegradable Polymers, edited by Domb, Kost and Wisemen, Hardwood Academic Press, 1997, pages 251-272. Copoly(ether-esters) for the purpose of this invention include those copolyester-ethers described in Journal of Biomaterials Research, Vol. 22, pages 993-1009, 1988 by Cohn and Younes and Cohn, Polymer Preprints (ACS Division of Polymer Chemistry) Vol. 30(1), page 498, 1989 (e.g. PEO/PLA). Polyalkylene oxalates for the purpose of this invention include U.S. Pat. Nos. 4,208,511; 4,141,087; 4,130,639; 4,140,678; 4,105,034; and 4,205,399 (incorporated by reference herein). Polyphosphazenes, co-, ter- and higher order mixed monomer based polymers made from L-lactide, D,L-lactide, lactic acid, glycolide, glycolic acid, para-dioxanone, trimethylene carbonate and ε-caprolactone such as are described by Allcock in The Encyclopedia of Polymer Science, Vol. 13, pages 31-41, Wiley Intersciences, John Wiley & Sons, 1988 and by Vandorpe, Schacht, Dejardin and Lemmouchi in the Handbook of Biodegradable Polymers, edited by Domb, Kost and Wisemen, Hardwood Academic Press, 1997, pages 161-182 (which are hereby incorporated by reference herein). Polyanhydrides from diacids of the form HOOC—C₆H₄—O—(CH₂)ₘ—O—C₆H₄—COOH where m is an integer in the range of from 2 to 8 and copolymers thereof with aliphatic alpha-omega diacids of up to 12 carbons. Polyoxaesters polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213 and 5,700,583; (which are incorporated herein by reference). Polyorthoesters such as those described by Heller in Handbook of Biodegradable Polymers, edited by Domb, Kost and Wisemen, Hardwood Academic Press, 1997, pages 99-118 (hereby incorporated herein by reference).

Moreover, suitable polymers include naturally occurring or synthetic materials that are biologically compatible with bodily fluid and mammalian tissues. Polymeric biomolecules for the purpose of this invention include naturally occurring materials that may be enzymatically degraded in the human body or are hydrolytically unstable in the human body such as fibrin, fibrinogen, collagen, elastin, and absorbable biocompatable polysaccharides such as chitosan, starch, fatty acids (and esters thereof), glucoso-glycans and hyaluronic acid.

In some embodiments according to the present invention wherein the polymer is poorly permeable and bioerodible, the rate of bioerosion of the polymer is advantageously sufficiently slower than the rate of drug release so that the polymer remains in place for a substantial period of time after the drug has been released, but is eventually bioeroded and resorbed into the surrounding tissue. For example, where the device is a bioerodible suture comprising the drug suspended or dispersed in a bioerodible polymer, the rate of bioerosion of the polymer is advantageously slow enough that the drug is released in a linear manner over a period of about three to about 14 days, but the sutures persist for a period of about three weeks to about six months. Similar devices according to the present invention include surgical staples comprising a prodrug suspended or dispersed in a bioerodible polymer.

In other embodiments according to the present invention, the rate of bioerosion of the polymer is advantageously on the same order as the rate of drug release. For instance, where the system comprises a drug or prodrug suspended or dispersed in a polymer that is coated onto a surgical implement, such as an orthopedic screw, a stent, a pacemaker, or a non-bioerodible suture, the polymer advantageously bioerodes at such a rate that the surface area of the prodrug that is directly exposed to the surrounding body tissue remains substantially constant over time.

In some embodiments according to the present invention, the polymer is non-bioerodible. Examples of non-bioerodible polymers useful in the present invention include poly(ethylene-co-vinyl acetate) (EVA), polyvinylalcohol and polyurethanes, such as polycarbonate-based polyurethanes. In other embodiments of the present invention, the polymer is bioerodible. Examples of bioerodible polymers useful in the present invention include polyanhydride, polylactic acid, polyglycolic acid, polyorthoester, polyalkylcyanoacrylate or derivatives and copolymers thereof. In yet other embodiments of the present invention, the polymer matrix is a mixture of both bioerodible and non-erodible polymers. The skilled artisan will recognize that the choice of bioerodibility or non-bioerodibility of the polymer depends upon the final physical form of the system, as described in greater detail below. Other exemplary polymers include polysilicone and polymers derives from hyaluronic acid. The polymer may be selected so that it will be the principal rate determining factor in the release of the drug or prodrug from the polymer.

Suitable non-bioerodible polymers with relatively low chronic tissue response, such as polyurethanes, silicones, poly(meth)acrylates, polyesters, polyalkyl oxides (polyethylene oxide), polyvinyl alcohols, polyethylene glycols and polyvinyl pyrrolidone, as well as, hydrogels such as those formed from crosslinked polyvinyl pyrrolidinone and polyesters could also be used. Other polymers could also be used if they can be dissolved, cured or polymerized on the stent. These include polyolefins, polyisobutylene and ethylene-alphaolefin copolymers; acrylic polymers (including methacrylate) and copolymers, vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile, polyvinyl ketones; polyvinyl aromatics such as polystyrene; polyvinyl esters such as polyvinyl acetate; copolymers of vinyl monomers with each other and olefins, such as ethylene-methyl methacrylate copolymers, acrylonitrile-styrene copolymers, ABS resins and ethylene-vinyl acetate copolymers; polyamides, such as Nylon 66 and polycaprolactam; alkyd resins; polycarbonates; polyoxymethylenes; polyimides; polyethers; epoxy resins, polyurethanes; rayon; rayon-triacetate, cellulose, cellulose acetate, cellulose acetate butyrate; cellophane; cellulose nitrate; cellulose propionate; cellulose ethers (i.e., carboxymethyl cellulose and hydroxyalkyl celluloses); and combinations thereof. Polyamides for the purpose of this application would also include polyamides of the form —NH—(CH₂)ₙ—CO— and NH—(CH₂)ₓ—NH—CO—(CH₂)_{y}—CO, wherein n is preferably an integer in from 6 to 13; x is an integer in the range of form 6 to 12; and y is an integer in the range of from 4 to 16. The list provided above is illustrative but not limiting.

Non-bioerodible polymers are especially useful where the system includes a polymer intended to be coated onto, or form a constituent part, of a surgical implement that is adapted to be permanently, or semi-permanently, inserted or implanted into a body. In certain embodiments, the surgical implement may consist entirely of the polymer. Exemplary devices in which the polymer advantageously forms a permanent coating on a surgical implement include an orthopedic screw, a stent, a prosthetic joint, an artificial valve, a permanent suture, a pacemaker, etc.

In certain embodiments, the polymers used for coatings have molecular weights high enough as to not be waxy or tacky. The polymers preferably adhere to the stent and are readily deformable after deposition on the stent as to be able to be displaced by hemodynamic stresses. The polymers molecular weight be high enough to provide sufficient toughness so that the polymers will not to be rubbed off during handling or deployment of the stent and not crack during expansion of the stent, though cracking can be avoided by careful placement of the coating, e.g., on portions of the stent which do not change shape between expanded and collapsed forms. The melting point of the polymer used in the present invention should have a melting temperature above 40° C., preferably above about 45° C., more preferably above 50° C. and most preferably above 55° C.

These polymers may be formed into film when advantageous.

In an exemplary embodiment, the nucleoside analog (or a prodrug or codrug thereof) may be incorporated into a polyfluoro copolymer comprising an amount of a first moiety selected from the group consisting of polymerized vinylidenefluoride and polymerized tetrafluoroethylene, and an amount of a second moiety other than the first moiety and which is copolymerized with the first moiety, thereby producing the polyfluoro copolymer, the second moiety being capable of providing toughness or elastomeric properties to the polyfluoro copolymer, wherein the relative amounts of the first moiety and the second moiety are effective to provide the coating and film produced therefrom with properties effective for use in treating implantable medical devices.

In certain embodiments, the device, e.g., a stent, may have two or more coatings, not all of which include the nucleoside analog (but rather other pharmaceutically active agents), or different concentrations of the nucleoside analog. For instance, the various coatings can differ in the concentration of prodrug, the identity of the prodrugs (active ingredients, linkers, etc), the characteristics of the polymer matrix (composition, porosity, etc) and/or the presence of other drugs or release modifiers.

In one exemplary embodiment, which can be useful where the drugs are provided as individual monomers rather than as codrugs, the polymeric matrix comprises two layers. The base layer comprises a solution of poly(ethylene-covinylacetate) and polybutylmethacrylate. The drug combination is incorporated into this base layer. The outer layer comprises only polybutylmethacrylate and acts as a diffusion barrier to prevent the drug combination from eluting too quickly. Other additives to modulate the release rate may also be incorporated in either or both layers. The thickness of the outer layer or top coat determines the rate at which the drug combination elutes from the matrix. Essentially, the drug combination elutes from the matrix by diffusion through the polymer matrix. Polymers are permeable, thereby allowing solids, liquids and gases to escape therefrom. The total thickness of the polymeric matrix is in the range from about one micron to about twenty microns or greater. It is important to note that primer layers and metal surface treatments may be utilized before the polymeric matrix is affixed to the medical device. For example, acid cleaning, alkaline (base) cleaning, salinization and parylene deposition may be used as part of the overall process described below.

To further illustrate, a poly(ethylene-co-vinylacetate), polybutylmethacrylate and drug combination solution may be incorporated into or onto the stent in a number of ways. For example, the solution may be sprayed onto the stent or the stent may be dipped into the solution. Other methods include spin coating and RF plasma polymerization. In one exemplary embodiment, the solution is sprayed onto the stent and then allowed to dry. In another exemplary embodiment, the solution may be electrically charged to one polarity and the stent electrically changed to the opposite polarity. In this manner, the solution and stent will be attracted to one another. In using this type of spraying process, waste may be reduced and more precise control over the thickness of the coat may be achieved.

The coatings may be of the same or different polymeric material. For example, a device may have a first coating that has low permeability, and a second coating, disposed on the first coating (which may or may not completely cover the first coating) that has high permeability. The first coating may include a nucleoside analog, such as 5-FU, that has high solubility in biological media, and the second coating may include a drug, such as TA, that has low solubility in biological media. Arranged in this way, the low-solubility agent, being in closer contact with the external environment, may be delivered into the environment at a rate similar to that of the high-solubility agent, the release of which is impeded by the second coating, whereas if the two agents were present in the same coating, the agent with the higher solubility would be released more rapidly than the less soluble agent.

In certain embodiments, the device, such as a stent, may be coated with a non-polymeric coating, preferably a porous coating, that includes (e.g., is impregnated with, or is admixed with) one or more pharmaceutically active compounds. Such coatings may include ceramic materials, organic materials substantially insoluble in physiologic fluids, and other suitable coatings, as will be understood by those of skill in the art. In certain other embodiments, the surface of the device itself is porous, e.g., the device may be formed of a porous material such as a ceramic or specially fabricated polymeric material, or the device may be formed in such a way that the surface achieves a porous character, and the pharmaceutically active compound is carried in the pores of the device's surface, thereby permitting gradual release of the compound upon introduction into a biological environment. In certain embodiments, the compound is 5-FU and/or TA. The surface of the device may further be coated with a polymeric material, e.g., that modulates the release of the agent(s), that improves biocompatibility, or otherwise improves the performance of the device in the medical treatment.

Another aspect of the invention relates to a device having a matrix, such as a fibrous matrix, such as a woven or non-woven cloth, e.g., vascular gauze (such as a Gortex® gauze), in which one or more pharmaceutically active compounds are disposed. In certain embodiments, the matrix is disposed on a stent, either wrapped around individual elements (e.g., wires) of the frame, or enveloping the entire device.

The coating of the present invention may be formed by mixing one or more suitable monomers and a suitable nucleoside analog agent, then polymerizing the monomer to form the polymer system. The nucleoside analog agent may be present as a liquid, a finely divided solid, or any other appropriate physical form. In this way, the agent is dissolved or dispersed in the polymer. In other embodiments, the agent is mixed into a liquid polymer or polymer dispersion and then the polymer is further processed to form the inventive coating. Optionally, the mixture may include one or more additives, e.g., nontoxic auxiliary substances such as diluents, carriers, excipients, stabilizers or the like. Other suitable additives may be formulated with the polymer and pharmaceutically active agent or compound. Suitable further processing includes crosslinking with suitable crosslinking agents, further polymerization of the liquid polymer or polymer dispersion, copolymerization with a suitable monomer, block copolymerization with suitable polymer blocks, etc. The further processing traps the drug in the polymer so that the drug is suspended or dispersed in the polymer coating.

In some embodiments, the nucleoside analog agent is incorporated into a biocompatible (i.e. biologically tolerated) polymer coating by dispersion. In some embodiments according to the present invention, the drug is present as a plurality of granules dispersed within the polymer coating. In such cases, it is preferred that the drug be relatively insoluble in the polymer coating, however the drug may possess a finite solubility coefficient with respect to the polymer coating and still be within the scope of the present invention. In either case, the polymer coating solubility of the drug should be such that it will disperse throughout the polymer coating, while remaining in substantially granular form.

In some embodiments according to the present invention, monomers for forming a polymer are combined with an inventive low-solubility compound and are mixed to make a homogeneous dispersion of the inventive compound in the monomer solution. The dispersion is then applied to a stent according to a conventional coating process, after which the crosslinking process is initiated by a conventional initiator, such as UV light. In other embodiments according to the present invention, a polymer composition is combined with an inventive low-solubility compound to form a dispersion. The dispersion is then applied to a stent and the polymer is cross-linked to form a solid coating. In other embodiments according to the present invention, a polymer and an inventive low-solubility compound are combined with a suitable solvent to form a dispersion, which is then applied to a stent in a conventional fashion. The solvent is then removed by a conventional process, such as heat evaporation, with the result that the polymer and inventive low-solubility drug (together forming a sustained-release drug delivery system) remain on the stent as a coating.

In some embodiments according to the present invention, the polymer is non-bioerodible, or is bioerodible only at a rate slower than a dissolution rate of the nucleoside analog agent, and the diameter of the granules is such that when the coating is applied to the stent, the granules' surfaces are exposed to the ambient tissue. In such embodiments, dissolution of the drug is proportional to the exposed surface area of the granules.

The drug or drug combinations may be incorporated onto or affixed to the stent in a number of ways. The inventive stent coating may be applied to the stent via a conventional coating process, such as impregnating coating, spray coating and dip coating. In the exemplary embodiment, the drug or drug combination is directly incorporated into a polymeric matrix and sprayed onto the outer surface of the stent. The drug or drug combination elutes from the polymeric matrix over time and enters the surrounding tissue. The drug or drug combination preferably remains on the stent for at least three days up to approximately six months, and more preferably between seven and thirty days.

In other embodiments according to the present invention, the system consists of a hard, solid polymer and forms a constituent part of a device to be inserted or implanted into a body, which is adapted to be inserted or implanted into a body by a suitable surgical method. In any of these embodiments, the polymer may be formed into a functional shape that has a physical function in addition to the polymer serving as a reservoir for the drug. In particular embodiments according to the present invention, the device consists of a hard, solid polymer, which is shaped in the form of a surgical implement such as a surgical screw, plate, stent, etc., or some part thereof. In other particular embodiments according to the present invention, the polymer in which the drug is suspended forms a tip or a head, or part thereof, of a surgical screw. In other embodiments according to the present invention, the system includes a polymer that is in the form of a suture having the drug dispersed or suspended therein.

In embodiments according to the present invention wherein the device is a surgical implement into which the prodrug and polymer have been incorporated as a constituent part, the polymer is advantageously a solid having physical properties appropriate for the particular application of the device. For instance, where the device is a suture, the polymer will have strength and bioerodibility properties suitable for the particular surgical situation. Where the device is a screw, stent, etc, the polymer is advantageously a rigid solid forming at least part of the surgical implement. In particular embodiments according to the present invention, such as where the system is part of a prosthetic joint, the polymer advantageously is non-bioerodible and remains in place after the prodrug has been released into the surrounding tissue. In other embodiments according to the present invention, such as in the case of bioerodible sutures, the polymer bioerodes after release of substantially all the prodrug.

The following examples are intended to be illustrative of the disclosed invention. The examples are non-limiting, and the skilled artisan will recognize that other embodiments are within the scope of the disclosed invention.

### EXAMPLE 1

TC-32 is a compound comprising 5FU linked to triamcinolone acetonide (TA).

A mixture of 3.3 gm Chronoflex C(65D) (Lot# CTB-G25B-1234) dispersion containing 0.3 gm of Chronoflex C(65D) and 2.2 gm Chronoflex C(55D) (Lot# CTB-121B-1265) dispersion containing 0.2 gm of Chronoflex C (55D), both in dimethyl acetamide (DMAC) (1:10, w/w) was prepared by mixing the two dispersions together. To this mixture, 6.0 gm of tetrahydrofurane (HPLC grade) were added and mixed. The final mixture was not a clear solution. Then 101.5 mg of TC-32 was added and dissolved into the polymer solution.

Ten (10) HPLC inserts were then coated with the polymer/TC-32 solution by dipping, which was then followed by air-drying under ambient temperature. The coating and air-drying process was repeated four (4) times (5 times total) until a total of about 10 mg of polymer/CT-32 was applied to each insert. The inserts were then placed in an oven at 80° C. for two hour to remove the residue of the solvent.

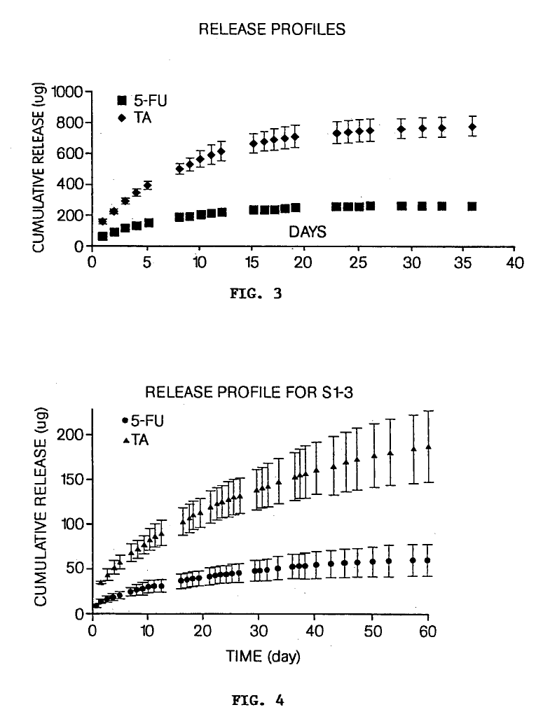

The inserts were placed individually in 20 ml of 0.1 m phosphate buffer, pH 7.4, in glass tube and monitoring of the release of compounds from the inserts at 37° C. was begun. Samples were taken daily, and the entire media was replaced with fresh media at each sampling time. The drugs released in the media were determined by HPLC. Because of the short half-life of TC-32 in buffer, no TC-32 was detectable in the release media; only amounts of parent drugs, 5FU and TA, could be determined. The release profiles are displayed in FIG. 3.

### EXAMPLE 2

To 5.0 gm of stirred dimethyl acetamide (DMAC), 300 mg of Chronoflex C(65D) (Lot# CTB-G25B-1234) and 200 mg of Chronoflex C(55D) (Lot# CTB-121B-1265) were added. The polymer was slowly dissolved in DMAC (about 4 hours). Then 5.0 gm of THF was added to the polymer dispersion. The mixture was not a clear solution. Then 100.9 mg of TC-32 was added and dissolved in the mixture.

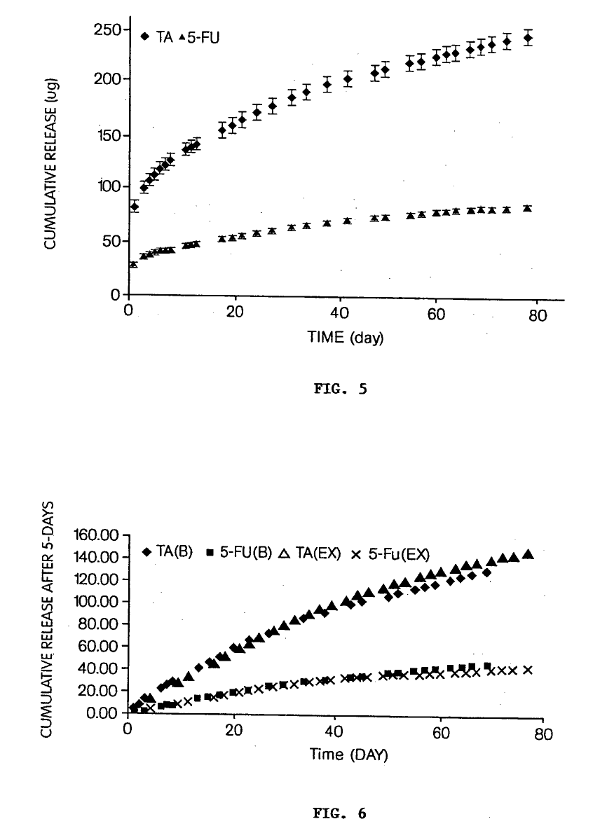

Three (3) Stents, supplied by Guidant Corp, were coated then with the polymer/TC-32 solution by dipping and followed by air-drying under ambient temperature. The coating and air-drying process was repeated a few times till a total of about 2.0 mg of polymer/TC-32 were applied to each stent. The coated stents were air-dried under ambient temperature in a biological safety cabinet over night. The stents were then vacuum dried at 80° C. for two hour to remove the residue of the solvent. Afterwards they were placed individually in 5.0 ml of 0.1 m phosphate buffer, pH 7.4, in glass tube and monitoring of the release of compounds from the stents was at 37° C. was begun. Samples were taken daily, and the entire media was replaced with a fresh one at each sampling time. The drugs released in the media were determined by HPLC. The release profiles were shown in the FIG. 4. No TC-32 was detectable in the release media.

The purpose of the above description and examples is to illustrate some embodiments of the present invention without implying any limitation. It will be apparent to those of skill in the art that various modifications and variations may be made to the systems, devices and methods of the present invention without departing from the spirit or scope of the invention. All patents and articles cited herein are specifically incorporated herein in their entireties.

### EXAMPLE 3

Chronoflex C (65D, Lot# CTB-G25B-1234) was first dissolved in tetrahydrofuran. Into this solution bioreversible conjugates of 5FU and TA were dissolved and the resulting solution spray coated onto coronary Tetra stents produced by Guidant. After air-drying, the coated stents were vacuum dried at 50° C. for 2 hours to remove solvent residue, and subject to plasma treatment and gamma-irradiation. Two different levels of drug loading were applied to stents: 80 ug Low Dose (13%) and 600 ug High Dose (60%). The release rate was determined in vitro by placing the coated stents (inflated with a dialation catheter: 3.0 mm balloon size and 20 mm long) in 0.1M phosphate buffer (pH 7.4) at 37° C. Samples of the buffer solution were periodically removed for analysis by HPLC, and the buffer was replaced to avoid any saturation effects.

The results shown in FIG. 5 illustrate the release pattern in vitro for a High Dose coated stent. The pattern followed a pseudo logarithmic pattern with approximately 70% being released in 10 weeks. A similar pattern is seen in both High Dose and Low Dose loaded stents. TA and FU were released in an equimolar fashion at all times during the experiments. No codrugs of 5FU/TA were detectable in the release media.

### EXAMPLE 4

Chronoflex C (65D; Lot# CTB-G25B-1234, 1.008 gm) was added to 50.0 gm of tetrahydrofuran (THF). The mixture was stirred overnight to dissolve the polymer. 5.0 gm of the polymer solution was diluted with 10.0 gm of THF. 150.2 mg of a codrug TC-32 (5-fluorouracil and triamcinolone acetonide) was added to the polymer solution and dissolved. The coating solution was prepared with 60% codrug loading. A 13% codrug loaded coating solution was also prepared. Bare stents (Tetra, Guidant, Lot# 1092154, 13 mm Tetra) were washed with isopropanol, air-dried, and spray coated with the coating solution using a precision airbrush. The coating was repeated until approximately 1.0 mg of total coating had been applied to each stent. After air-drying, the coated stents were vacuum dried at 50° C. for 2 hours to remove solvent residue, and subject to plasma treatment and gamma-irradiation

Codrug coated stents were tested in two groups. After inflated with a dialation catheter (3.0 mm balloon size and 20 mm long), Group One stents were placed individually into a glass tube containing 5.0 ml of 0.1 M phosphate buffer (pH 7.4). Samples were taken periodically and the concentration of codrug in the buffer was tested by HPLC. The entire release media was replaced after each sample.

Group Two stents were placed in vivo. Three common swine had TC-32 coated stents implanted into the left anterior descending (LAD) coronary artery on study day 1. The stents were harvested on study day 5 and then placed in 0.1 M phosphate buffer as describe for Group One stents. The amount of each drug released into the media was determined by HPLC. The intact codrug was not detectable in release media.

The results are shown in FIG. 6, showing the comparative drug release profiles between explanted stents and non-implanted stents. The release patterns for both explanted and pre-implanted stents; indicate that in-vivo release may be predicted by in vitro release patterns.

### EXAMPLE 5

Fourteen (14) domestic swine received a maximum of three (3) stents deployed in any of the three-epicardial coronaries (LAD, LCX, and RCA). Some animals were given only control stents, comprising either Bare Metal Tetra Coronary Stent on Cross Sail Rx balloon delivery system (Control), or PU Coated Tetra Coronary Stent on Cross Sail Rx balloon delivery system (Control). Other animals were given drug-coated stents either in Low Dose (80 μg TA+5FU (13%)) or High Dose (600 μg TA+5FU (60%)). The stents were implanted into arteries of the animals. Each stent was advanced to the desired location in the artery, and was deployed using an inflation device. The pressure of the inflation device was chosen to achieve a balloon to artery ratio of 1.1-1.2:1.

After 28 days, arterial sections directly adjacent to the stents were surgically excised and embedded in a methacrylate resin. Histologic 5-μm sections were cut and stained with Verhoeffs elastin and Hematoxylin and Eosin stains, and the thickness of each excised section was measured. The results are shown in table for both High and Low Dose drug-coated stents. The response at 28 days in both low-dose and high-dose experimental groups shows a profound reduction in intimal thickness attributed to the co-release of TA and 5FU3 from polymer coated Tetra stents

| | Bare Metal | Polymer | Low Dose | High Dose |
|---|---|---|---|---|
| Balloon:artery | 1.07 ± 0.05 | 1.11 ± 0.07 | 1.13 ± 0.05 | 1.11 ± 0.08 |
| ratio | | | | |
| Intimal | 0.29 ± 0.03 | 0.36 ± 0.08 | 0.13 ± 0.01^{ξ} | 0.13 ± 0.04 [symbol] |
| Thicnkess | | | | |
| (mm) | | | | |
| Medial area | 1.39 ± 0.10 | 1.98 ± 0.41 | 0.96 ± 0.06^{§} | 0.98 ± 0.07 [symbol] |
| (mm²) | | | | |

^{ξ}p = 0.0008 Bare Metal vs. Low Dose, p = 0.03 Polymer vs. Low Dose
^{§}p = 0.002 Bare Metal vs. Low Dose, p = 0.04 Polymer vs. Low Dose [symbol]
p = 0.02 Bare Metal vs. High Dose, p = 0.07 Polymer vs. High Dose [symbol]
p = 0.01 Bare Metal vs. High Dose, p = 0.07 Polymer vs. High Dose

### EXAMPLE 6

Stents were coated with a mixture of TA and 5FU in a mole-ratio of 1 to 1 without chemical linkage. The release rate was determined in vitro by placing the coated stents in 0.1M phosphate buffer (pH 7.4) at 37° C. Samples of the buffer solution were periodically removed for analysis by HPLC, and the buffer was replaced to avoid any saturation effects.

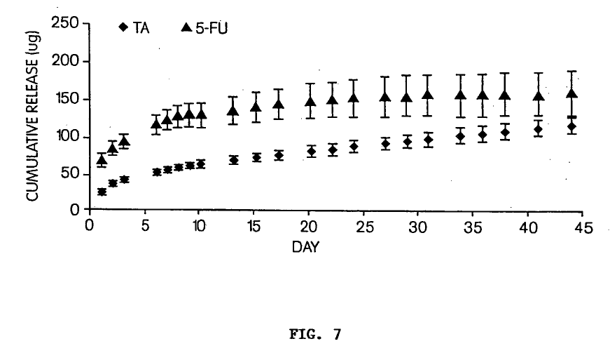

The results are shown in FIG. 7. Because of the hydrophilic nature of 5FU, this compound was released from the mixture coating much faster than from the codrug coating. Within 4 weeks, more than 95% of total 5FU was released. TA release from the drug mixture coating was much slower, with about 20% TA released over the first 6 weeks.

The 5FU/TA mixture in a polymer coating demonstrated different release profiles compared to the codrug polymer coating. However, this study indicates that use of a mixture of 5FU and TA can be applied to a stent to achieve controlled release of a desired active compound mixture.

### EXAMPLE 7

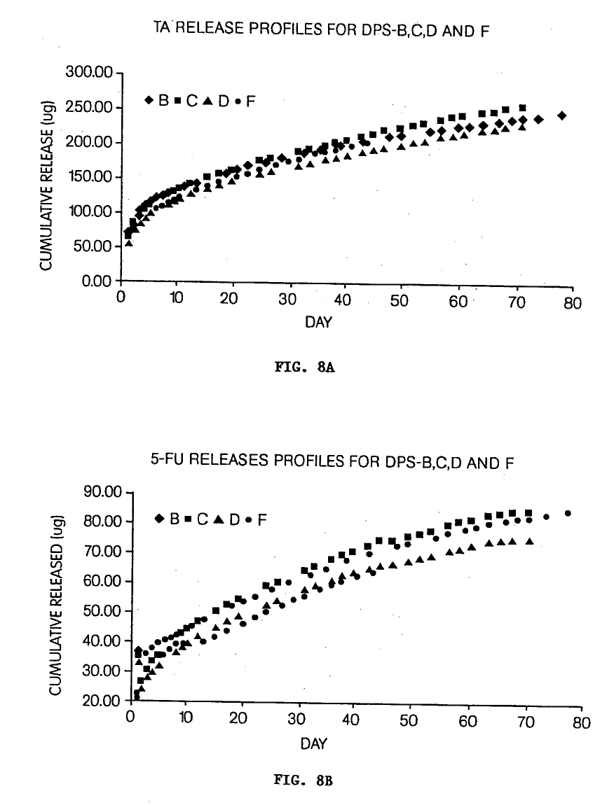

A polymer-coated stent was also tested to identify any inherent release pattern attributable to the polymer. Following plasma treatment and gamma-irradiation, the stents were inflated with a dilatation catheter (3.0 mm balloon size, 20 mm long) and placed individually into a glass tube containing 5.0 ml of 0.1 M phosphate buffer (pH 7.4). Samples were taken periodically and the entire release media was replaced after each sample. The amount of each drug released into the media was determined by HPLC. The intact codrug was not detectable in release media. See FIGS. 8A and 8B.

We claim:

1. A medical device comprising:
(a) a substrate having a surface; and
(b) a coating disposed on the surface, said coating comprising a polymer matrix including a nucleoside analog, or a codrug or prodrug thereof, which nucleoside analog is formulated to produce an effective concentration of said nucleoside analog in tissue or biological fluid in which the medical device is implanted over a period of at least 15 days.

2. A medical device comprising:
(a) a substrate having a surface; and
(b) a coating disposed on the surface, said coating comprising a polymer matrix including a soluble nucleoside analog, and one or more additives which decrease the rate of release of the nucleoside analog into the biological fluid or tissue surrounding the device,
wherein the nucleoside analog is released from said polymer matrix at a rate of release to produce an effective concentration of said nucleoside analog in tissue or biological fluid in which the medical device is implanted.

3. A medical device comprising:
(a) a substrate having a surface; and
(b) a coating disposed on the surface, said coating comprising a polymer matrix including a prodrug/codrug, wherein said prodrug/codrug is represented by the general formula A—L—B, in which
A represents a nucleoside analog or a prodrug thereof;
L represents a covalent bond or covalent linker linking A and B to form the prodrug/codrug, wherein the bond or linker is metabolized under physiological conditions; and
B represents a moiety which, when linked to A, results in a compound having an optimized solubility for sustained release of said nucleoside analog from the coated device at the implanted site.

4. A medical device comprising:
(a) a substrate having a surface; and
(b) a coating disposed on the surface, said coating comprising a polymer matrix including a reduced solubility prodrug/codrug, wherein said prodrug/codrug is represented by the general formula of A::B, in which
A represents a nucleoside analog or a derivative thereof;
:: represents an ionic bond between A and B that dissociates under physiological conditions to generate said pharmaceutically active form of A; and
B represents a moiety which, when linked to A, results in a compound having an optimized solubility for sustained delivery in vivo from the coated device.

5. A medical device comprising:
(a) a substrate having a surface; and
(b) a coating disposed on the surface, said coating comprising a polymer matrix including 5-fluorouracil.

6. The device of any of claims 1-4, wherein the nucleoside analog is a fluorinated pyrimidine or purine analog, or a prodrug thereof.

7. The device of any one of claims 1-5, wherein the polymer is non-bioerodible.

8. The device of any one of claims 1-5, wherein the polymer is bioerodible.

9. The device of claim 8, wherein the bioerodible polymer contains polyanhydride, polylactic acid, polyglycolic acid, polyorthoester or polyalkylcyanoacrylate, or derivatives or copolymers thereof.

10. The device of any one of claims 3-4, wherein A and B are the same nucleoside analog moieties.

11. The device of any one of claims 3-4, wherein B, after cleavage from the prodrug/codrug, is a biologically or pharmacologically inactive moiety.

12. The device of any one of claims 3-4, wherein B is selected from immune response modifiers, anti-proliferatives, anti-mitotic agents, anti-platelet agents, platinum coordination complexes, hormones, anticoagulants, fibrinolytic agents, anti-secretory agents, anti-migratory agents, immunosuppressives, angiogenic agents, angiotensin receptor blockers, nitric oxide donors, antisense oligionucleotides and combinations thereof, cell cycle inhibitors, corticosteroids, angiostatic steroids, anti-glaucoma drugs, antibiotics, differentiation modulators, antiviral drugs, anticancer drugs, and anti-inflammatory drugs.

13. The device of claim 6, wherein said nucleoside analog is a fluorinated pyrimidine.

14. The device of claim 13, wherein the nucleoside analog is selected from 5-fluorouracil (5-FU), 5′-deoxy-5-fluorouridine, 5-fluorouridine, 2′-deoxy-5-fluorouridine, fluorocytosine, 5-trifluoromethyl-2′-deoxyuridine.

15. The device of claim 13, wherein the fluorinated pyrimidine is 5-fluorouracil (5-FU) or a prodrug thereof.

16. The device of any one of claims 3-4, wherein B is a non-steroidal anti-inflammatory.

17. The device of claim 24, wherein B is selected from diclofenac, fenoprofen, flurbiprofen, ibuprofen, ketoprofen, ketorolac, nabumetone, naproxen and piroxicam.

18. The device of claim 3, wherein the linkage L includes one or more hydrolyzable groups selected from an ester, an amide, a carbamate, a carbonate, a cyclic ketal, a thioester, a thioamide, a thiocarbamate, a thiocarbonate, a xanthate and a phosphate ester.

19. The device of claim 3, wherein the linkage L is enzymatically cleaved.

20. The device of any of claims 3 or 4, wherein the prodrug/codrug has a logP value at least 0.5 logP units more than the logP value for nucleoside analog.

21. The device of any of claims 3 or 4, wherein the prodrug/codrug has a solubility in water at 25° C. at least 50 percent less than the solubility of the nucleoside analog.

22. The device of claim 3 or 4, wherein the prodrug/codrug has a solubility less than 0.1 mg/mL in water at 25° C.

23. The device of any of claims 1-5, wherein the polymer reduces interactions, when implanted, between the nucleoside analogs in the polymer and proteinaceous components in surrounding biological fluid.

24. The device of any of claims 1-5, wherein said coating provides sustained release of a therapeutically effective amount of the nucleoside analog for a period of at least 30 days.

25. The device of any one of claims 1-5, wherein the substrate is a surgical implement selected from a screw, a plate, a washer, a suture, a prosthesis anchor, a tack, a staple, an electrical lead, a valve, a membrane, an anastomosis device, a vertegral disk, a bone pin, a suture anchor, a hemostatic barrier, a clamp, a clip, a vascular implant, a tissue adhesive or sealant, a tissue scaffold, a bone substitute, an intraluminal device and a vascular support.

26. The device of any one of claim 1-5, wherein the substrate is selected from catheters, implantable vascular access ports, blood storage bags, blood tubing, central venous catheters, arterial catheters, vascular grafts, intraaortic balloon pumps, heart valves, cardiovascular sutures, artificial hearts, a pacemaker, ventricular assist pumps, extracorporeal devices, blood filters, hemodialysis units, hemoperfusion units, plasmapheresis units, filters adapted for deployment in a blood vessel, intraocular lenses, shunts for hydrocephalus, dialysis grafts, colostomy bag attachment devices, ear drainage tubes, leads for pace makers and implantable defibrillators, and osteointegrated orthopedic devices.

27. The device of any one of claims 1-5, which is a vascular stent.

28. A method for treating a mammalian organism to obtain a desired local or systemic physiological or pharmacological effect, comprising: administering a pharmaceutically effective amount of a drug by placing in said mammal the device of any of claims 1-5 to a mammal.

29. A method for treating an intraluminal tissue of a patient, the method comprising the steps of:
(a) providing the stent of claim 27;
(b) positioning the stent at an appropriate intraluminal tissue site; and
(c) deploying the stent.

30. A method of manufacturing a coating for a medical device, comprising admixing a polymer matrix and a pharmaceutically effective amount of a nucleoside analog, or a codrug or prodrug thereof, which nucleoside analog is formulated in a form having a solubility less than 0.1 mg/mL in water at 25° C.

31. Use of a polymeric coating in the manufacture of a device to place in a patient for treatment of said patient with a sustained dosage regimen of a nucleoside analog, which nucleoside analog is formulated in a form having a solubility less than 0.1 mg/mL in water at 25° C.
